(19) United States
(12) Patent Application Publication
(10) Pub. No.: US 2026/0237496 A1
(43) Pub. Date: Aug. 13, 2026

(54) METHOD AND SYSTEM FOR INTELLIGENT PATIENT PLANNING AND SCHEDULING IN DYNAMIC HEALTHCARE ENVIRONMENT

(71) Applicant: IMAM MOHAMMAD IBN SAUD ISLAMIC UNIVERSITY, Riyadh (SA)

(72) Inventors: Aïssa REZZOUG, Riyadh (SA); Saif ULLAH, Taxila (PK); Aisha TAYYAB, Taxila (PK); Nashmi H. ALRASHEEDI, Riyadh (SA); Mirza JAHANZAIB, Taxila (PK); Wasim AHMAD, Taxila (PK); Salman HUSSAIN, Taxila (PK)

(73) Assignee: IMAM MOHAMMAD IBN SAUD ISLAMIC UNIVERSITY, Riyadh (SA)

(21) Appl. No.: 19/053,295

(22) Filed: Feb. 13, 2025

(51) Int. Cl. G16H 40/20

(52) U.S. Cl. CPC G16H 40/20

(57) ABSTRACT

A method and system for a healthcare facility includes predicting, by machine learning models for healthcare provider availability prediction, material resource prediction, machinery condition and availability probability prediction, and patient health data prediction, values of each variable for a future time. Different simulation models are used to identify critical resource in a planning period in advance and select an appropriate dedicated model of the identified critical resource, that are subject to constraints and optimization objectives, determine various feasible solutions of a planning and scheduling algorithm at various levels of a planning hierarchy. Plans are synchronized with a patient plan and schedule which is generated for various levels of planning hierarchy. Push and pull rules are used to move patients between planning periods for generation of feasible plans. An optimal patient plan and schedule of patients is generated, released and implemented in the healthcare computing network.

### BACKGROUND

### Technical Field

The present disclosure is directed to a method and system to generate, execute and implement optimized patient plan and schedule on resources in a healthcare facility. The optimal plan generated by the method and system is integrated with a maintenance plan and schedule for machinery of the healthcare facility. Moreover, the optimized patient plan and schedule is also integrated with a material requirement plan and material release schedule and a human resource plan and schedule.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

The initial cost and operational cost of healthcare facilities have significant contribution from the surgical units and their interconnected resources including operating theater complex (OT), preoperative holding units (PHU), post anesthesia care unit (PAHU), intensive care unit (ICU), and wards in the healthcare facility.

The operating theater complex contains multiple operating rooms to perform surgeries of specialized procedures. Underutilization of these resources directly increases the operational cost of the healthcare facility and reduces the service level. Efficient planning and scheduling of patients and healthcare resources is significant to improve healthcare performance with respect to various optimization objectives. Inefficient planning and scheduling might occur due to lack of consideration of constraints which exist between the interconnected resources of healthcare facilities.

Many healthcare facilities prepare the plan and schedule of patients on resources in advance by shifts, days, weeks, hours with simple methods. The plan generated with these simple methods may include the plan and schedule of doctors, surgeons, operating rooms, radiology, wards, consumable items and staff. These plans are mostly prepared independently for simplicity and the plans are not prepared in integrated manner and may include all interrelated constraints. However, a simple or manual method of creating patient plan and schedule on resources is time consuming and does not take into consideration all variables which are affecting the patient plan. Simple and Manual methods make the calculations easier but do not timely integrate all variables while creating plans and schedules of patients and resources. The plan generated with simple calculations and manual methods generates a sub-optimal plan due to lack of interrelated constraints consideration during planning or sometimes makes an infeasible plan when executed which negatively affects the service level and increases cost of operation.

Different systems and methods for planning and scheduling of healthcare facilities have been developed. In US2002/0131572A1 a system and method for scheduling appointments of patients and the schedule required for resources is disclosed. The disclosed system has limited scope and focuses on the patient appointment related issues in the healthcare facility.

In EP2060986A1 a system and method for management of processes in a hospital/operating room is disclosed. A modular system, which is computer-based, is provided for management of operating room related tasks.

In U.S. Pat. No. 7,562,026B2 a user interface system for operational and resource information of radiology information system is disclosed. The disclosed radiology resource monitoring system comprises at least one repository maintaining data identifying scheduled procedures, data identifying room and equipment availability and data identifying clinician availability. The invention concerns a system for monitoring radiology resources and patient status and tracking patients and the progress of procedures for the patients.

In U.S. Pat. No. 8,311,850B2 a method is disclosed to schedule resources to deliver healthcare services to patients. The method identifies availability of a series of resources, calculates predicted duration to deliver healthcare service to each patient, calculates start and end time of block of time and its probability for which resources will not be available, calculates probability that resources will be available in a block of time and generates an optimal schedule with confidence level. The disclosed method generates a schedule with confidence level and risk.

In US20120203564A1 a computer implemented method and system for real-time optimal management of hospital's emergency room resources is disclosed. The disclosed system provides an optimal work plan for crew members and equipment, taking into consideration relevant variables, and the method automatically updates the emergency room planning in real time.

In U.S. Pat. No. 8,452,615B2 a computer-implemented method for managing operating room resources in a hospital and a modular computer-based system for managing operating-room related processes in a hospital are disclosed.

In US20140039906A1 techniques for generating optimized surgery schedules have been disclosed. The developed technique receives a plurality of surgery requests in a surgery facility having plurality of operating rooms. The developed method identifies resource constraints, generates a surgery schedule for a surgery facility including sequencing and operating room assignment for each of the surgical procedures considering resource constraints and optimization objective. The developed method simulates the surgical schedule to determine the expected operational metrics associated with the surgical procedure.

In US20210193302A1 patient placement and sequencing method in dynamic environment is disclosed. The current state data of various medical facility systems including operating conditions of a medical facility system, patient case data of active cases and pending cases and other contextual data including workflow timings, patient occupancy level, and bed availability levels is used to predict the future state data using a machine learning framework. The disclosed method employed a heuristic based optimization method to determine optimal reactive solution regarding patient sequencing, patient placement and resource allocation based on current state information, future state information, rules and optimization criteria. The method uses current state data of the system in a forecasting component where processing time of different cases of patients is predicted and resource demand is predicted for the future, using machine learning techniques. Once the process time and resource requirements are predicted, the optimization component of the disclosed method generates an optimal sequence of patients and resource allocation based on an optimization objective. The disclosed method generates patient placement, patient sequence and timing and resource allocation which is executed in the hospital in real time.

In US20210193302A1 conventional techniques have considered historic information and utilized machine learning techniques to determine the future state information of a system and use it to generate a schedule of patients and resources. However, all variables which can influence the resources and their possibility of becoming critical in future planning periods and ultimately can influecne future planning and scheduling of hospital resources is significant to be considered for advance planning and scheduling of patients and resources. Without current status, real time status and historic information of all types of variables including but not limited to human resources, patients, material, equipment/machinery, community, weather and climate, events, and the supply chain of a healthcare facility, which can influence directly or indirectly on the advance planning and scheduling of patients, resources, material and equipment/machinery, the schedule of patients generated by US20210193302A1 may not be effective and therefore considered in the present invention.

In most of the healthcare facilities, the large quantity of the real time information of various variables including but not limited to historic data and real time status data real time condition data of patients, human resources, machinery and material, increases the complexity of the data processing and computing, but can be handled through cloud computing technology and information technology (IT) to transfer data from patients, material, machinery, human resources and all other resources, community, weather and climate, events and incidences, supply chain of healthcare facility and surroundings through sensors/devices/computers to a cloud data storage. Cloud data storage is a significant centralized information and data storage which can increase robustness and improve the timely delivery of data to the system.

Cloud data storage is used when a large quantity of data is required to be stored at a centralized location so that the data can be used by several processes which are running in parallel and needs the data at the same time for their processing.

Cloud computing techniques have been used in the biological environment to store, analyze and access the real time biological data as described in the document US2013/0275486 and it can be used for data storage and data retention in an industrial automation application as described in the document US 2010/0257228 A1.

Thus, conventional healthcare systems used in healthcare facilities are mainly focused on registering arriving patients, tracking their use of various resources, and using automatic billing systems, to streamline the flow of patients' use of resources. These systems are mostly working as hospital management systems which are significant for the current operations of the healthcare facility, and they create one kind of feasible plans and schedules. The state of the artwork presented the systems which are mainly aimed at developing patient appointments (US2002/0131572A1). Moreover, there are some other systems disclosed in the state of the artwork for management of operating rooms to give optimal plan of patients on operating rooms (EP2060986A1; US20140039906A1). In addition, some disclosures of healthcare management systems have developed patient plans on radiology resources and can these systems give an information system of radiology department of healthcare facility (U.S. Pat. No. 7,562,026B2). Furthermore, there are disclosures in the state of artwork which are focused on developing a schedule of surgeries in operating rooms or patient plans in the emergency rooms (US20120203564A1). Moreover, few of the state of artwork disclosed patient planning systems which used historic data and machine learning methods for prediction of values of some of the variables which affect patient planning and scheduling (US20210193302A1). However, state of the art has not considered data of all variables which can influence patient planning and scheduling. For example, state of artwork has not considered all the data from historic data of variables related to patients, resources, material, machinery maintenance, climate, weather, events and incidences and supply chain of healthcare facilities. Moreover, the existing systems are not intelligent enough to make optimal future plan and schedule of patients on resources in advance of the planning periods. Most of the existing systems create feasible schedule and do not optimized these schedules and plans in advance of planning periods.

The existing healthcare systems are creating schedules of patients based on their arrival pattern and propose one kind of feasible plan and schedule. However, these systems are not intelligent enough to create precise plans and schedules in advance before the execution of plans and schedules in planning periods. Resources plans for, human resources, material and machinery maintenance plans are prepared separately from patient planning using deterministic approaches with fixed planning cycles in most of the existing management systems. These approaches are not robust and need changes by reactive planning and scheduling when uncertain parameters of system changes including uncertain machine failures, uncertain shortage of some kind of material which has been planned based on deterministic usage rate. These kinds of issues ultimately affect the patient's waiting times and there is always a need for overtime staff, uncertain maintenance activities, uncertain delays due to shortage of materials. The existing systems may not create significant integration between patient planning and scheduling with human resources, material planning and resource maintenance planning and scheduling. Due to lack of integration, there is possibility that in some planning periods, a critical resource may be changed in planning periods, or resources of hospital may remain idle due to lack of human resources, material or due to maintenance operations and in some planning periods, the similar resources may be overloaded, and overtime works are performed on them to manage patients. Moreover, the planning and scheduling in these systems is performed in hierarchical manner in which higher level plan of healthcare facility is fixed, once it is made, it is communicated to medium level planning decisions and lower-level panning decisions in hierarchical manner. There is a lack of real time feedback information mechanism which can communicate the change of critical resource, or communicate the change in system conditions and this may limit these systems to only plan and schedule the patients which are already freezing in the relevant higher level planning periods. These systems have less flexibility due to which resources are not managed optimally and plans and schedules are not optimal. Patients need to wait in long queues and resources are not utilized at maximum capacity in planning periods. In addition, the existing system does not take into consider the variables of human fatigue, learning, forgetting and ergonomics aspects during making patient plan. In addition, existing systems are not considering the variables including forecast of variables related to weather and climate, community, events and incidences supply chain of healthcare facilities for intelligent and accurate prediction of patients in the planning periods. Moreover, the existing systems are not accurate enough to intelligently estimate the expected values of variables and parameters which are related to human resources, machines, material, surroundings, weather and climate, supply chain. Therefore, existing systems are not intelligent enough to prepare patient plans and schedule on resources and existing systems have limited integration with material requirement plan, material release plan and schedule, maintenance plan and human resource plan in dynamic healthcare facility.

Accordingly, it is one object of the present disclosure to provide methods and system for integrating patient planning and scheduling with material planning and maintenance planning and scheduling and human resource planning in an intelligent manner in advance of planning periods in a dynamic environment of healthcare facility. A further object is to maximize utilization of critical resources and other resources of a healthcare facility in planning periods in an intelligent and efficient manner.

### SUMMARY

An aspect is an intelligent system for patient planning and scheduling in a dynamic healthcare environment of a healthcare facility, that can include a healthcare facility computing network; a healthcare facility data base management system maintaining historical data related to status and condition of healthcare provider resources, material, machinery and patients; and a process module having processing circuitry configured to retrieve the historical data from the healthcare facility data base, and real time data, corresponding to a plurality of variables related to the status and condition of healthcare provider resources, material, machinery and patients, to predict, by a plurality of machine learning models for healthcare provider availability prediction, material resource prediction, machinery availability probability prediction, and patient health data prediction, that are updated using the retrieved historical data, values of each variable for a future time based on the real time and historical data, and input the predicted values of all variables to an advance planning and scheduling engine. The advanced planning and scheduling engine is configured, with different simulation models that are subject to constraints and optimization objectives, to determine a critical resource in the planning periods, run the planning and scheduling model of the identified critical resource and determine various feasible solutions of a planning and scheduling algorithm at various levels of a planning hierarchy and determine an optimal plan and schedule of patients. The processing circuitry is further configured to generate an optimal material requirement plan and schedule, an optimal material release plan and schedule, an optimal machinery maintenance plan and schedule, and an optimal healthcare provider resource plan and schedule in advance; synchronize the plans with a patient plan and schedule which is generated for various levels of planning hierarchy, use push and pull rules to move patients between planning periods for generation of feasible plans, perform iterations of an optimization algorithm to search optimal patient plan, integrated with human resource plan, material plan, resources plan and resources maintenance plan, generate the optimal patient plan and schedule of patients on resources integrated with an optimal maintenance planning and scheduling of machinery, an optimal material requirement planning, material release scheduling and an optimal human resource planning and scheduling in a dynamic environment of healthcare facility to create an integrated plan and schedule, and release and implement an optimal integrated plan and schedule in the healthcare computing network.

A further aspect is a computer-implemented method for patient planning and scheduling in a dynamic healthcare environment of a healthcare facility having a healthcare computing network. The method can include obtaining real time and retrieving historical data, from a healthcare facility data base, corresponding to a plurality of variables related to status and condition of healthcare provider resources, material, machinery and patients; predicting, by a plurality of unidentical machine learning models for healthcare provider availability prediction, material resource prediction, machinery availability probability prediction, and patient health data prediction, that are updated using the retrieved historical data, values of each of the variables for a future time based on the real time and historical data; inputting the predicted values of the variables to an advance planning and scheduling engine, wherein the advanced planning and scheduling engine is configured, with multiple unidentical simulation models that are subject to constraints and optimization objectives; determining critical resource in the coming planning period, generating various feasible solutions of the simulation models of the planning and scheduling algorithm at various levels of a planning hierarchy; generating a material requirement plan and schedule, material release plan and schedule, machinery maintenance plan and schedule, and healthcare provider resource plan and schedule in advance; synchronizing the plans with a patient plan and schedule which is generated for various levels of the planning hierarchy; using push and pull rules to move patients between planning periods for generation of feasible plans; performing iterations of an optimization algorithm to search an optimal patient plan, integrated with a human resource plan, a material plan, a resources plan and a resources maintenance plan; generating the optimal patient plan and schedule of patients on resources integrated with maintenance planning and scheduling of machinery, material requirement planning, material release scheduling and human resource planning and scheduling in a dynamic environment of healthcare facility to create an integrated plan and schedule; and releasing and implementing the integrated plan and schedule in the healthcare computing network.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1 illustrates a block diagram of the elements in an example, non-limiting system for optimization of the integrated patient plan and schedule on resources with an optimal human resource plan and schedule on resources, with an optimal material requirement and material release plan and schedule on resources and an optimal resource maintenance plan and schedule of machinery and executes it dynamically in the healthcare facility with one or more embodiments of the disclosed subject matter;

FIG. 2 illustrates a high-level overview of modules comprised in the disclosed subject matter;

FIG. 3 illustrates block diagram of the input module of the disclosed subject matter;

FIG. 4 illustrates block diagram of data base/management system in the input module of the disclosed method and system;

FIG. 5 illustrates block diagram of real time status and condition in the input module of the disclosed method and system;

FIG. 6 illustrates block diagram of real time and past information from surroundings in the input module of the disclosed method and system;

FIG. 7 illustrates block diagram of the process module of the disclosed subject matter;

FIG. 8 illustrates block diagram of the estimation engine in the process module of the disclosed method and system;

FIG. 9 illustrates schematic diagram of integration of input module and process module in the disclosed method and system;

FIG. 10 illustrates block diagram of the advance planning and scheduling engine in the process module of the disclosed method and system;

FIG. 11 illustrates block diagram of the output module of the disclosed subject matter;

FIG. 12 illustrates schematic diagram of integration of input module, process module, execution and cloud data in the disclosed method and system;

FIG. 13 illustrates a schematic view of the system and method comprising of modules and the working of each module of the system;

FIG. 14 is an illustration of a non-limiting example of details of computing hardware used in the computing system, according to certain embodiments.

FIG. 15 is an exemplary schematic diagram of a data processing system used within the computing system, according to certain embodiments.

FIG. 16 is an exemplary schematic diagram of a processor used with the computing system, according to certain embodiments.

FIG. 17 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Some of the embodiments disclosed herein describe basic concepts and are merely illustrative in the context of one or more components of the present disclosure which can be modified for different applications with modifications in algorithms, optimization objectives, constraints, type of modeling approaches through simulations or other methods of models. Various components presented here can be implemented in any of various known manners, for example, by any programming language, any software, automation, (programmable, using internet, wireless, sensors, actuators), firmware, and so on, or any combination of these protocols. In one embodiment, one of the implementations of the components is described. In other embodiments, other single components are described with the description of separate components to indicate their individual function. There is no intention to be bound by any expressed or implied information described herein, including the preceding Summary section or in the Detailed Description of Embodiment section.

Aspects of this disclosure are directed to a method and system, apparatus and/or software and hardware system that provide and intelligently generate and executes an optimal plan and schedule of patients on various resources of the healthcare facility in dynamic environment integrating with an optimal plan and schedule of human resources, optimal material requirement plan, optimal material release plan and schedule and optimal maintenance plan and schedule of resources and take into consideration historic and current and real time data of all variables from various sources including but not limited to data base/management system of healthcare facility, real time status and condition of resources and real time and past data and information from surroundings, community, weather and climate, events and incidences, supply chain of healthcare facility to generate estimated values of the variables of the system and utilizing models with objectives, constraints, simulation, heuristics and algorithms.

The disclosure relates to predicting the future state of timeline forecast, resource demand forecast, patient sequence and timing solution, patient placement solution and resource allocation solution and prepared an optimal schedule of patients and resource allocation. However, in a dynamic environment of healthcare facilities, the identification of a critical resource before the planning period time is significant in order to make an efficient and intelligent plan and prepare resources before a planning period for which the plan is prepared. The resources may have different number and type of patients in different planning periods as per future predictions. In addition, resources, workers on the resources, and material may have different conditions and status in the planning periods. Due to different conditions and status of resources, material, machines and human resources in planning periods, a critical resource may be a different resource in different time frames and planning periods. Moreover, a resource to become a critical resource also depends on the type of patients arriving in the healthcare facility, estimated times of patients on various resources, condition of all resources and their real time status, condition of human resources and their real time status etc. Therefore, when changing the values of variables of human resources, material, machinery and patients in different planning periods, the critical resource also changes in planning periods. However, the resource can become critical in planning periods and the machine learning based models as developed in US20210193302A1 may not be efficient to determine the critical resource in dynamic environment. The present invention identifies critical resources in a dynamic environment of a healthcare facility considering estimated values of all variables of human resources, materials, machinery and patients. The invention includes models to determine a critical resource from all resources in dynamic environment.

Moreover, the plan and schedule made on the critical resource also depends on the type of resource which becomes critical, as every resource has different working conditions and process methods and therefore, every critical resource has its own dedicated model to make a patient plan on it and have its own dedicated model to calculate its efficiency. The prediction models as presented in US20210193302A1 only relies on the performance of prediction algorithms to determine future state variables. However, the present disclosure not only estimates the values of variables of human resources, materials, machinery and patients, but also determines the resource which will become critical resource in the coming planning period in advance of time by days, weeks, months etc. Present disclosure finds an optimal plan and schedule on the identified critical resource using its own dedicated advance planning and scheduling heuristic and its own dedicated model. Moreover, the present invention also takes into consideration of interconnected constraints of all interconnected resources and all interconnected variables to determine a best feasible plan and schedule.

The method and system integrates the variables and constraints of human resources, patients, material, resources of healthcare facility, machinery, community, events and incidences, weather and climate, surroundings and supply chain of healthcare facilities. The method and system generate an integrated plan of patients in an intelligent manner by predicting most expected values of all variables related to human resources, patients, material, resources of healthcare facility, machinery, community, events and incidences, weather and climate, surroundings and supply chain of healthcare facilities. These values are significant to calculate the resource becoming critical in the coming planning period in advance and hence an appropriate and dedicated model with defined objectives and set of constraints and algorithm is executed to determine an optimal plan and schedule on the identified critical resource which is also linked with the start and end time of different patients on various interlinked resources and hence helps in creating the most realistic plans and schedule. In addition, the rules, heuristics, models, and algorithms are new and can predict the most expected constraint resource in the near coming planning period in advance for proactive planning and scheduling of patients in the planning periods. The method and system can generate all feasible plans and schedules which are accepted by all constraints of human resources, patients, material, resources of healthcare facility, machinery, community, events and incidences, weather and climate, surroundings and supply chain of healthcare facilities and determine most optimal plan and schedule and communicate it to all planning and scheduling levels in real time to update the status of all resources. In addition, it can identify and utilize critical resource of the coming planning period at maximum and generate optimal patient plan and schedule, case mix plan, an optimal configuration of operating rooms, optimal master surgery schedule, and optimal allocation schedule and advance schedule of patients and communicate patient plan and schedule on resources, human resource plan and schedule on resources, material requirement and material release plan and schedule on resources and resource maintenance plan and schedule of machinery in advance of the planning period. As will be described below, Criticality of resources in medium level planning (MLP) and Look ahead planning periods are calculated. In addition, the system and method execute and implement an optimal patient plan and schedule on resources, executes optimal human resource plan and schedule on resources, executes optimal material requirement and material release plan and optimal schedule on resources, executes optimal resource maintenance plan and schedule on machinery in the near future planning period scale using different actuators in the healthcare facility in real time. In addition, system and method synchronizes all plans with the patient plan and schedule which is generated for the various levels of planning hierarchy. The system and method integrates all levels of planning hierarchy with a feedback mechanism at all levels of the planning hierarchy and use push and pull rules for generation of feasible solutions for various iterations in the optimization algorithms. The disclosed method and system inputs the processed information to the cloud-based data storage utilizing any kind of data sharing medium and also sends the processed data with the output module.

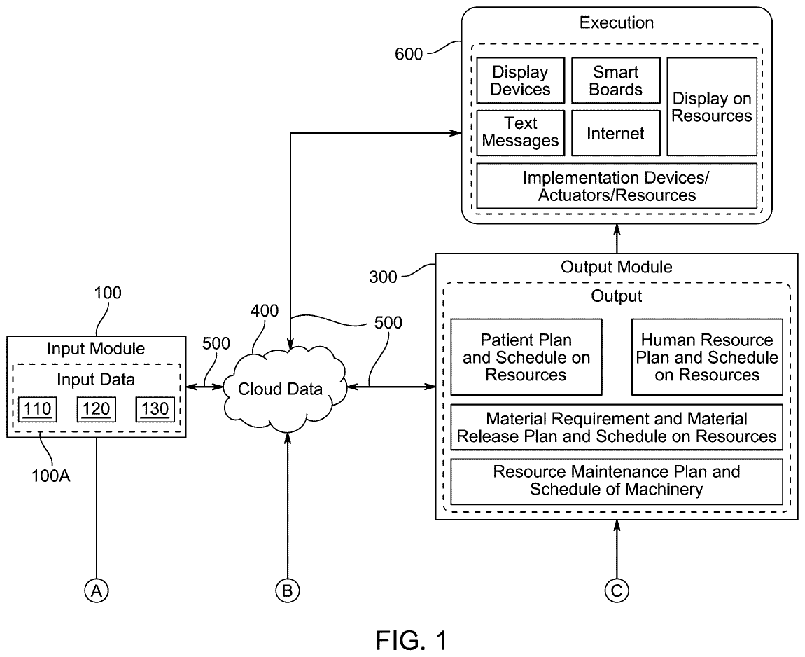

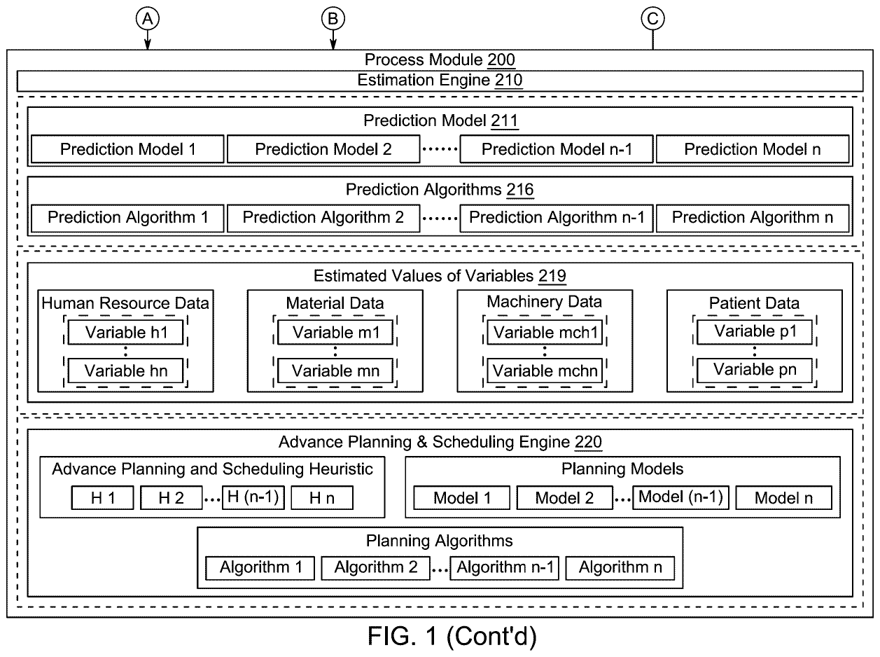

FIG. 1 illustrates a block diagram of the disclosed method and system 1. The method and system 1 comprises input module 100, process module 200, output module 300, cloud-based data storage 400, information sharing mechanism 500 and execution mechanism 600. The input data 100A is obtained from the resources of a healthcare facility and surroundings through different kinds of devices including but not limited to RFID sensors, websites, location sensors, health monitoring devices to measure real time data of various variables and historic data of variables from the healthcare facility and surroundings which is shared with the cloud-based data storage 400 through an information sharing mechanism 500 including but not limited to internet, WIFI, Bluetooth, and wireless network. Cloud-based data storage 400 stores the input data 100A and shares it with the other connected modules including process module 200 and output module 300 utilizing an information sharing mechanism 500 in real time. The process module offloads the required data of variables from cloud-based data storage 400 utilizing information sharing mechanism 500 to process and generate estimated values of the variables 219 using estimation engine 210 and prediction algorithms 216.

The estimated values of the variables are shared with the advance planning and scheduling engine 220 where advance planning and scheduling heuristics 221, models 222 and algorithms 223 are utilized to generate and share optimal plan and schedule of patients on various resources 310, optimal human resource plan and schedule on resources 320, optimal material requirement and optimal material release plan and schedule on resources 330 and optimal resource maintenance plan and schedule on machinery, and share it with the cloud-based data storage 400 and output module 300 in real time utilizing information sharing mechanism 500. The output module 300 offloads data from cloud-based data storage 400 of optimal plan and schedule of patients on various resources 310, optimal human resource plan and schedule on resources 320, optimal material requirement and material release plan and schedule on resources 330 and optimal resource maintenance plan and schedule on machinery and shares it with execution mechanism 600 where these plans are executed and implemented on the healthcare facility network dynamically in real time utilizing various actuators including but not limited to display of schedules on display devices, smart boards, shared through internet, mobile phone messages, generation of work plan for various resources and displays and run on computer systems in real time with all resources.

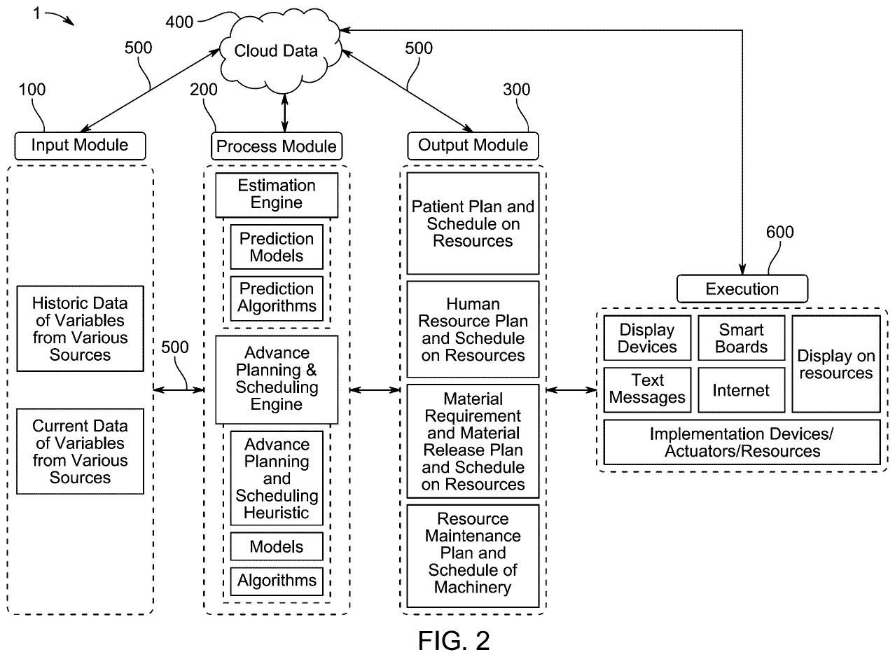

FIG. 2 illustrates high level overview of the system and method. The high level system and method comprises input module 100, process module 200, output module 300, cloud-based data storage 400, information sharing mechanism 500 and execution 600. The input module 100 comprises historic data of variables obtained from various sources and current data of variables obtained from various sources. The input module 100 shares this data with cloud-based data storage 400 utilizing an information sharing mechanism 500 including but not limited to internet, computer systems integration, WIFI, data sharing and smart devices. The process module 200 offloads the required data of variables from cloud-based data storage 400 utilizing information sharing mechanism 500 including but not limited to internet, computer integrated systems. The process module 200 comprising estimate engine 210 and advance planning and scheduling engine 220 where optimal integrated planning and scheduling of patients on resources, optimal human resource plan and schedule on resources, optimal material requirement and material release plan and schedule on resources and resource optimal maintenance plan and schedule of machinery is generated utilizing estimated values of variables 219 which are calculated in the estimation engine 210. These plans are stored in cloud-based data storage 400 and output module 300 utilizing the information sharing mechanism 500 in real time. The output module 300 releases an optimal plan and schedule of patients on resources, an optimal human resource plan and schedule on resources, an optimal material requirement and material release plan and schedule on resources, and resource optimal maintenance plan and schedule of machinery with all resources in advance and stores in the cloud-based data storage 400. The execution mechanism 600 offloads optimal plans from cloud-based data storage 600 and executes and implements optimal integrated plans in the healthcare facility using various methods including but not limited to display devices, smart boards, internet, display on resources, and actuators.

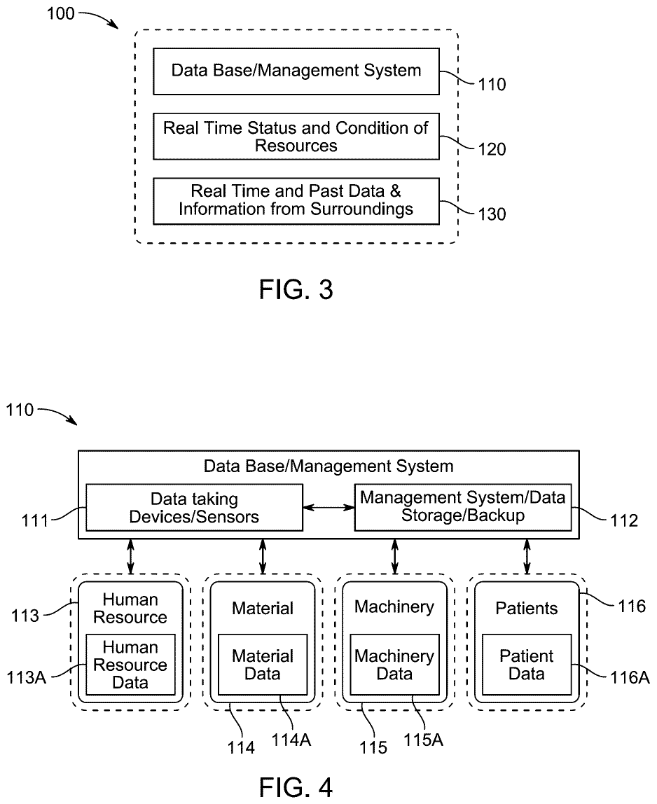

FIG. 3 illustrates block diagram of the input module of the disclosure. The input module comprising input data of various variables from sources including but not limited to a data base management system 110, real time status and condition of resources 120, and real time and past data and information from surroundings 130.

For purposes of this disclosure, resources in a healthcare facility relate to departments and units and wards within the healthcare facility, including but not limited to operating theater complex (OT), preoperative holding units (PHU), post anesthesia care unit (PAHU), intensive care unit (ICU), and wards in the healthcare facility. Machinery in a healthcare facility relates to various medical equipment, including but not limited to equipment in an operation theater, equipment in an intensive care unit, equipment in preoperative holding units, equipment in post anesthesia care units, equipment in wards, laboratory equipment, an X-ray facility, radiology equipment, MRI, CT-Scan, in the healthcare facility. Material in a healthcare facility relates to any materials used for healthcare, including but not limited to vaccines, medicines, blood, instruments, oxygen supply, oxygen cylinders, injections, cotton, gauze.

Human resources, some of the machinery, and materials may be assigned to departments or units or wards in the healthcare facility. For purposes of this disclosure, human resources, machinery, and material are referred to as “on resources,” meaning being assigned to particular departments or units of the healthcare facility.

FIG. 4 illustrates block diagram of data base management system 110 in the input module of the disclosed method and system. The data is obtained utilizing data taking devices 111 and management system/data storage or backup data 112 of the healthcare facility from various sources including human resource 113, material 114, machinery 115 and patients 116. The human resource data 113A includes but is not limited to status and historic information of age, gender, experience, learning attitude, fatigue behavior, learning behavior, learning rate, forgetting behavior, forgetting rate, tasks performance and tasks performance attitude of doctors, nurses, and any other staff/human resource involved directly or indirectly in the operations of healthcare facility. The material data 114A includes but is not limited to current status and historic information of frequency of use of any kind of material, its deterioration, its replacements, expiry details, frequency of use and orders status, their time of arrivals, lead times of any form and type of material used in the healthcare facility including but not limited to vaccines, medicines, blood, instruments, oxygen supply, oxygen cylinders, injections, cotton, gauze, to name a few. Furthermore, the data includes data related to material use for machine maintenance, and tools used for machine maintenance. The machinery data 115A includes but is not limited to current status and historic information of any form and type of failure of machinery used in the healthcare facility, current status and historic information of machinery of any kind and type used which is used in the healthcare facility including but not limited to: operation theater, intensive care unit, preoperative holding units, post anesthesia care units, wards, laboratories, X-ray facility, radiology, MRI, CT-Scan, and real time status and condition of any form and type of equipment and its parts, machinery used and its working parts status in healthcare facility. Patient's data 116A includes but not limited to past information of number and type of patients of different gender and age group arrived or admitted in the hospital, time history of various patients, type of diseases, type of operations, duration of operations, to name a few.

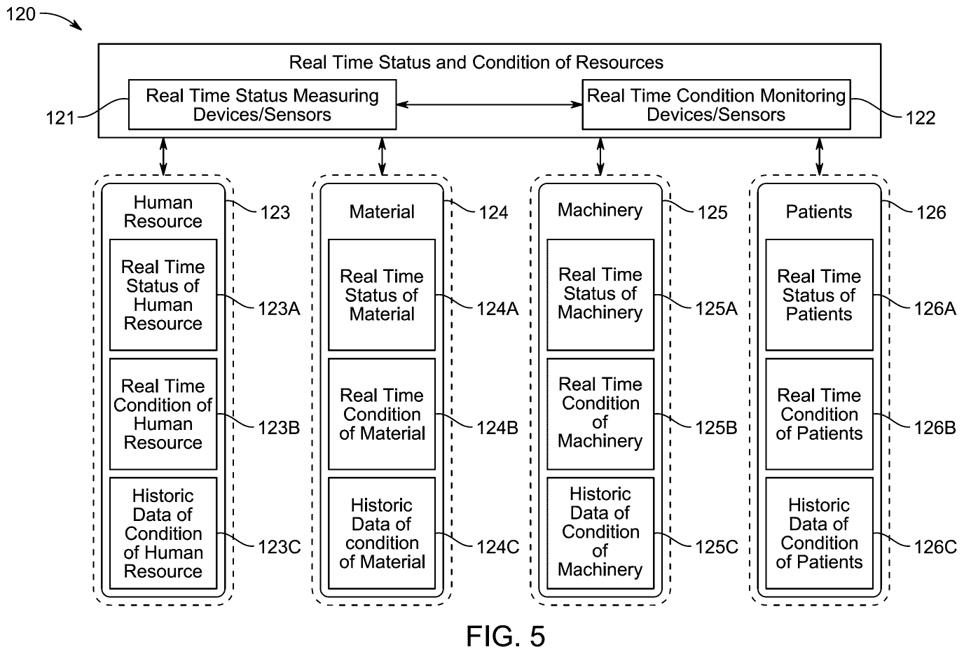

FIG. 5 illustrates a block diagram of real time status and condition of resources 120 in the input module of the disclosed method and system 1. The real time status and condition of resources 120 comprises real time status measuring devices/sensors 121 and real time condition monitoring devices/sensors 122. The real time status measuring devices/sensors 121 and real time condition monitoring devices/sensors 122 are utilized to get real time status and condition of human resources 123, material 124, machinery 125, and patients 126.

The real time status and condition of human resources 123 are utilized to get real time status of human resources 123A, real time condition of human resources 123B, and historic data of condition of human resources 123C. The status, condition, and historic data of human resources includes but not limited to real time health status, experience, learning, forgetting, fatigue, tasks performance status, and tasks performance attitude, of doctors, nurses, and other staff/human resource involved directly or indirectly in the operations of healthcare facility, to name a few. The status and condition of human resources may be obtained from or calculated based on a health practitioners profile, records of hours on duty, and sick days, as entered by the practitioners in computer terminals connected to the healthcare facility computer network.

The real time status measuring devices/sensors 121 and real time condition monitoring devices/sensors 122 are utilized to get real time status of various kinds of material 124A, real time condition of various kinds of material 124B, historic data of condition of various kinds of material 124C. The status, condition, and historic data of material used in healthcare facilities includes but not limited to real time status and condition of medicines, blood, instruments, injections, cotton and original and current shelf life. The status of materials may be monitored by scanners, for example bar code scanners or the like, that scan materials whenever they are replenished and whenever they are dispensed. The scanners may be connected to computer terminals located throughout the healthcare facility. The computer monitors may be mobile devices, in which case the scanners are portable. The condition of materials may be measured by maintaining a count of a number of times of usage or maintaining a count of a quantity or volume of items being dispensed.

The real time status measuring devices/sensors 121 and real time condition monitoring devices/sensors 122 are utilized to get real time status of various types or kinds of machinery 125A, real time condition of various types or kind of machinery 125B, historic data of condition of various types or kinds of machinery 125C. The status, condition, and historic data of various forms and types of machinery include but are not limited to real time status and condition of any form and type of equipment and its parts, machinery used and its working parts status in healthcare facility, the real time working condition of the equipment used in different departments but not limited to: operation theater, Intensive care unit, wards, laboratories. The status and condition of machinery may be monitored using scanners that detect time and period of usage of a machine, so that reliability of the machine can be predicted.

The real time status measuring devices/sensors 121 and real time condition monitoring devices/sensors 122 are utilized to get real time status of patients 126A, real time condition of patients 126B and historic data of condition of patients 126C. The status, condition, and historic data of patients include but is not limited to the real time health status and condition, real time data of blood tests results of patients, test reports of various samples of patients, severity level of patient, real time blood pressure, real time sugar level, real time temperature, real time ECG, real time EEG data of admitted patients taken from various sensors/devices/lab tests/reports, diagnosis of patient and other patient-related methods. The status and condition of patients is made by entering health condition information in a computer terminal of the healthcare facility computer network. Patients can also enter personal information via a Web portal.

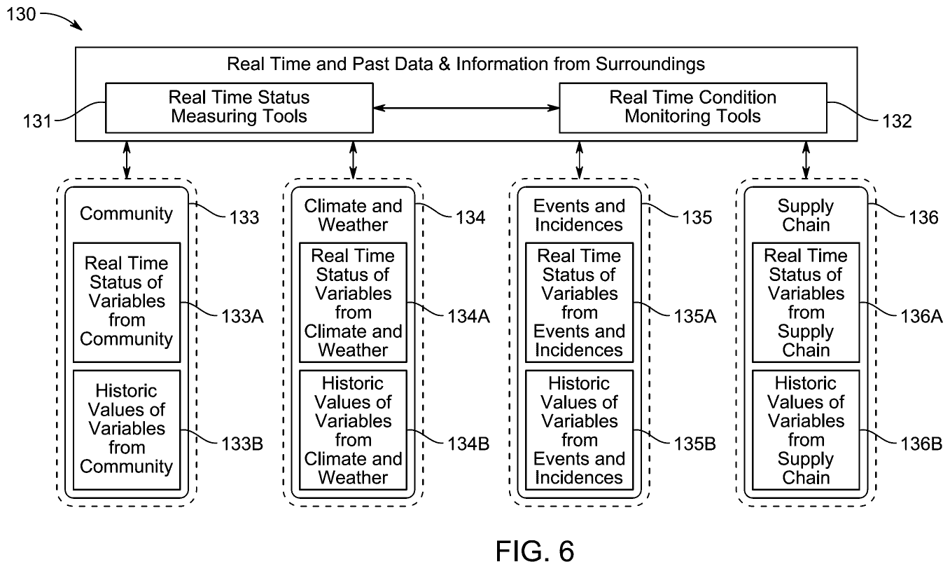

FIG. 6 illustrates a block diagram of real and past information from surroundings 130 of the disclosed method and system 1. The real and past information from surroundings 130 comprises real time status monitoring tools 131 including but not limited to websites, blogs, social media platforms, artificial intelligence tools, and real time condition monitoring tools 132 including but not limited to sensors, smart devices, cloud services, internet, internet of things, websites, social media. Real time status monitoring tools 131 and real time condition monitoring tools 132 are used to get real time status and real time condition of variables from community 133, real time status and real time condition of variables from climate and weather 134, real time status and real time condition of variables from events and incidences 135, real time status and real time condition of variables from supply chain of healthcare facility 136.

The real time status of variables from community 133A, and historic values of variables from community 133B includes but not limited to macro environment, microenvironment, demographic data, size, density, location, gender, race, culture, subculture, reference class, age, life stages, lifestyles, beliefs, attitudes.

The real time status of variables from climate and weather 134A and historic values of variables from climate and weather 134B includes but not limited to temperature, pressure, moisture, season, climate changes, water level, smoke, clouds, pollution, oxygen level, change in temperature, changes in pressure.

The real time status of variables from events and incidences 135A and historic values of variables from events and incidences 135B includes but not limited to political situation, season, fashion, festivals, gatherings, sports and recreational activities, protests, accidents, disasters including but not limited to flood, earthquake.

The real time status of variables from supply chain of healthcare facility 136A and historic values of variables from supply chain of healthcare facility 136B includes but not limited to condition and location of manufacturers, inventory level of suppliers, retailers, warehouses, of all kinds of material which is used by healthcare facilities like vaccine, surgical instruments, cotton and bandages, medicines, machinery tools, blood of various types, to name a few.

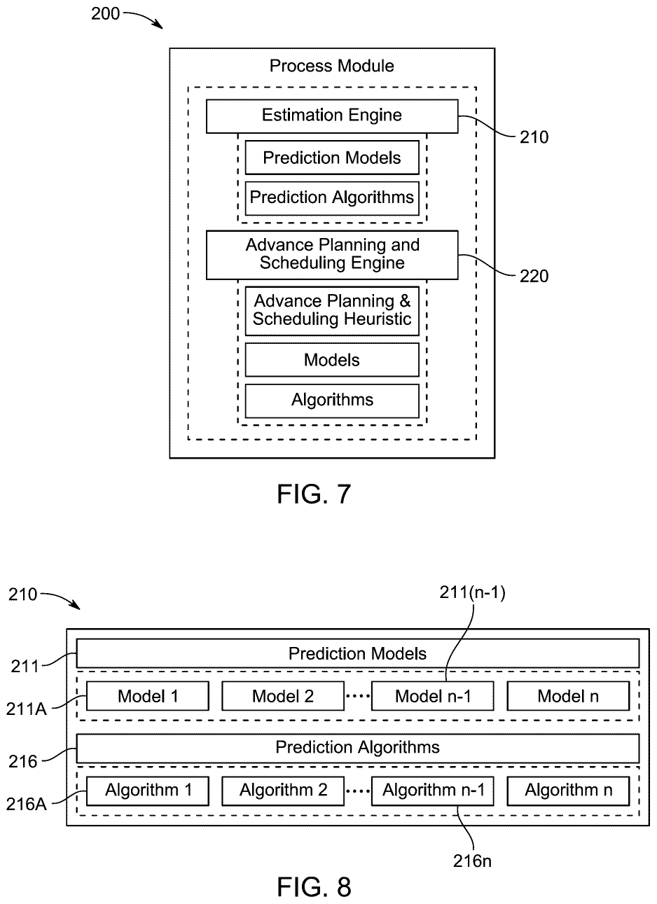

FIG. 7 illustrates block diagram of the process module 200 in the disclosed method and system 1. Process module 200 comprises estimation engine 210 and advance planning and scheduling engine 220. The estimation engine 210 includes prediction models and prediction algorithms. The advanced planning and scheduling engine 220 includes planning models and planning algorithms.

FIG. 8 illustrates a block diagram of the estimation engine 210 in process module 200 in the disclosed method and system 1. The estimation engine 210 comprises prediction models 211 and prediction algorithms 216. Prediction models 211 contain various machine learning models 211A, including but not limited to Model 1, Model 2, Model (n−1), Model (n) that operate in parallel. Prediction models 211A of the method and system 1 are for each variable which can influence directly or indirectly on the patient planning and scheduling in healthcare facility. The prediction models 211A includes a model for prediction of variable including but not limited to probability of failure of printer of X-ray machine, probability of failure of scanner of ultrasound machine, expected time of replacement of printer of X-ray machine, expected time required to replace the printer of X-ray machine, predicted probability of drowsiness of staff working in pathology lab, predicted value of tiredness level of staff taking blood samples from patients, predicted value of time of non-availability of a doctor, predicted value of number of cardiac patients arriving in near future planning period, expected severity of patient in ICU, to name a few.

The prediction algorithms 216 contains various training and machine learning algorithms 216A, including but not limited to Algorithm 1, Algorithm 2, Algorithm (n−1), Algorithm (n) that operate in parallel. These machine learning algorithms can be any of various supervised learning machine learning algorithms, including but not limited to decision trees, artificial neural networks, support vector machine. Prediction algorithms 216A in the method and system 1 for estimation of values of each variable which can influence directly or indirectly on the patient planning and scheduling in healthcare facility. The prediction algorithms 216A include training algorithms for prediction of variables including but not limited to probability of failure of printer of X-ray machine, probability of failure of scanner of ultrasound machine, expected time of replacement of printer of X-ray machine, expected time required to replace the printer of X-ray machine, predicted probability of drowsiness of staff working in pathology lab, predicted value of tiredness level of staff taking blood samples from patients, predicted value of time of non-availability of a doctor, predicted value of number of cardiac patients arriving in near future planning period, expected severity of patient in ICU, to name a few.

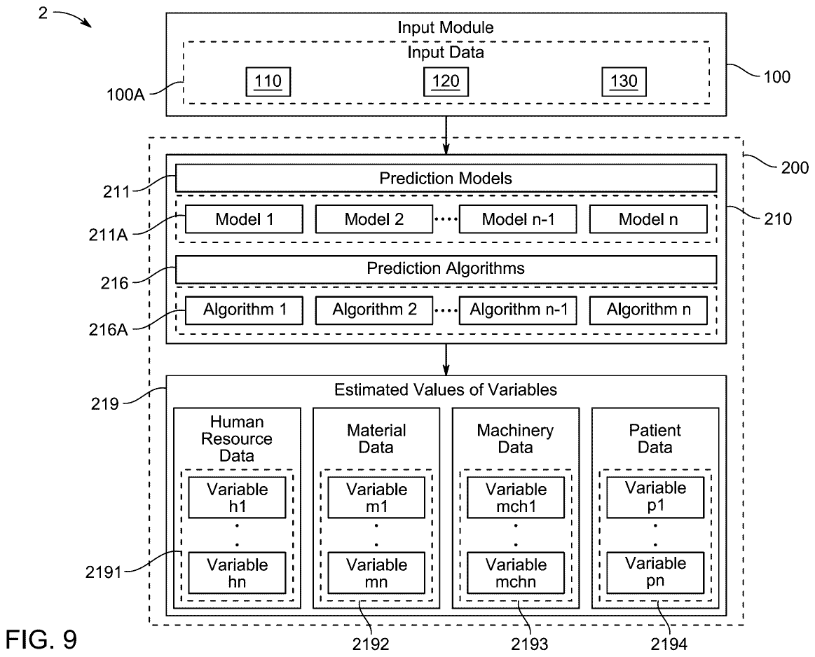

FIG. 9 illustrates schematic diagram of integration 2 of input module 100 and process module 200 in the disclosed method and system 1. Input data 100A in input module 200 containing data from database management system 110, data of real time status and condition of resources 120 and real time and past data and information from surroundings 130, is transferred to the process module 200.

The process module 200 includes prediction models 211 containing prediction models 211A for each variable, which are used to predict the value of variables utilizing prediction algorithms 216 for each variable 216A is used to get the estimated value of each variable 219.

The estimated values of variables 219 include the estimated values of variables related to human resource data 2191 including but not limited to predicted probability of drowsiness of staff working in pathology lab, predicted value of tiredness level of staff taking blood samples from patients, predicted value of time of non-availability of a doctor, to name a few. The estimated values of variables 219 includes an estimated value of each variable related to material data 2192 including but not limited to expected time of arrival of blood sample in blood bank, expected shortage of vaccine. The estimated values of variables 219 include and estimated value of each variable related to machine data 2193 including but not limited to probability of failure of printer of X-ray machine, probability of failure of scanner of ultrasound machine, expected time of replacement of printer of X-ray machine, expected time required to replace the printer of X-ray machine, to name a few. The estimated values of variables 219 includes an expected value of each variable related to patient data 2194 including but not limited to predicted value of number of cardiac patients arriving in near future planning period, expected severity of patient in ICU, to name a few.

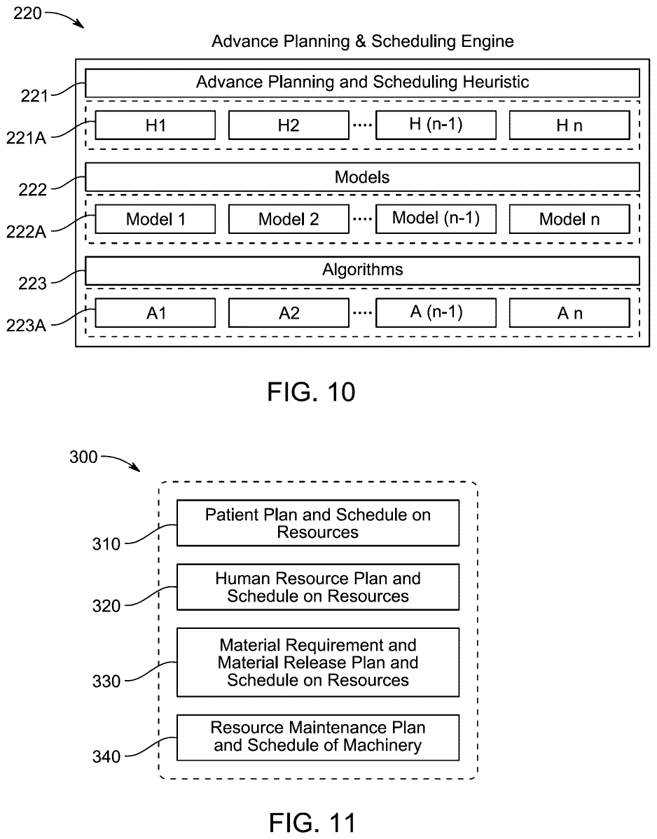

FIG. 10 illustrates block diagram of the advance planning and scheduling engine 220 in process module 200 of the disclosed method and system 1. The advance planning and scheduling engine 220 comprises advance planning and scheduling heuristics 221, models 222 and algorithms 223. The heuristics 221 contains planning and scheduling heuristics including but not limited to H1, H2, . . . , H(n−1), H(n), 221A, which are utilized to define various planning and scheduling conditions, rules, commands, if and else scenarios, and any other form and type of logics and priority methods or conditions, to name a few.

The models 222 contain plaining and scheduling models including but not limited to Model 1, Model 2, . . . , Model (n−1), M(n), 222A, which can be analytical, numerical, stochastic, programming models or any kind of simulation models in various simulation platforms, GUI, and/or software, and applications. The models 222A are proposed for all resources which can become critical in a planning period and each critical resource has a dedicated model with defined objective and set of constraints for different planning and scheduling problems including but not limited to infinite capacity planning and capacity allocation problem, capacity constraint identification model, case mix problem, operating room configuration problem, master surgery problem, allocation problem, advance scheduling problem with optimization objectives including but not limited to minimize makespan, maximize utilization, minimize waiting time of patients, maximize revenue, minimize overtime, maximize throughput, minimize tardiness, minimize cancelations, minimize cost or any other maximizing or minimizing objective and constraints including but not limited to capacity constraints of upstream and downstream wards and all departments and any kind of constraints of their resources, availability constraints of human resources, material, equipment and machines, patient constraints of earliness, lateness, number of patients, health life constraints of equipment, probability of failure constraints of machinery and their components, health life constraints of human resources, ergonomics constraints of all human resources, material related constraints of deterioration, material expiry constraints, material arrival constraints and their other related constraints.

The algorithms 223 contain planning and scheduling algorithms including but not limited to A1, A2, . . . , A(n−1), A(n), 223A, which are used to solve the models 222A and generate optimal/near optimal/best solutions in reasonable computational time. The optimization algorithms 223A contain algorithms including but not limited to solution methods based on any form and type of mathematical programming, exact methods, heuristics, simulation methods, analytical procedures, big data analytics, metaheuristics, reinforcement learning methods.

FIG. 11 illustrates a block diagram of the output module 300 of the disclosed method and system 1. The output module 300 comprises a patient plan and schedule on resources 310, human resource plan and schedule on resources 320, material requirement and material release plan and schedule on resources 330 and resource maintenance plan and schedule of machinery 340. The patient plan and schedule on resources 310 includes but not limited to the allocation of patients to different time slots on various resources, the expected start time and completion time of tasks on patients including but not limited to their operations/tests/diagnosis/surgery. Moreover, it includes the plan and schedule of patients on various resources in the healthcare facility describing their timings. The patient plan and schedule 310 on resource includes timing of patients in advance for coming months, weeks, days or any other time period unit used in the healthcare facility.

Human resource plan and schedule 320 include but not limited to the duty timings of all human resources in the healthcare facility on various resources in advance of month, week, days or any other kind of time period scale or unit used in the healthcare facility. The timing of duty involves but not limited to the start time, working time, finishing time of doctors, nurses, operating room assistants, anesthesiologist, consultants, X-ray machine operators, laboratory workers, to name a few.

Material requirements and material release plan and schedule 330 include the advance plan and schedule of any kind and type of material which is expected to be used in the healthcare facility in the near future in near coming month, week, day or other planning period scale or unit used in the healthcare facility. The material requirement plan includes but not limited to the time of use of any type and kind of material, its expected quantity, location of use, and condition of use. In addition, the material release plan of any type and kind of material to be used by any resource in the healthcare facility and its timings and location for different operations including but not limited to its loading, transportation, unloading, and arrival.

The resource maintenance plan and schedule of machinery 340 include but not limited to the time, location and type of requirement of maintenance of any kind and type of resource which is used in the healthcare facility in advance in the near future in the near coming month, week, day or other planning period scale or unit used in the healthcare facility.

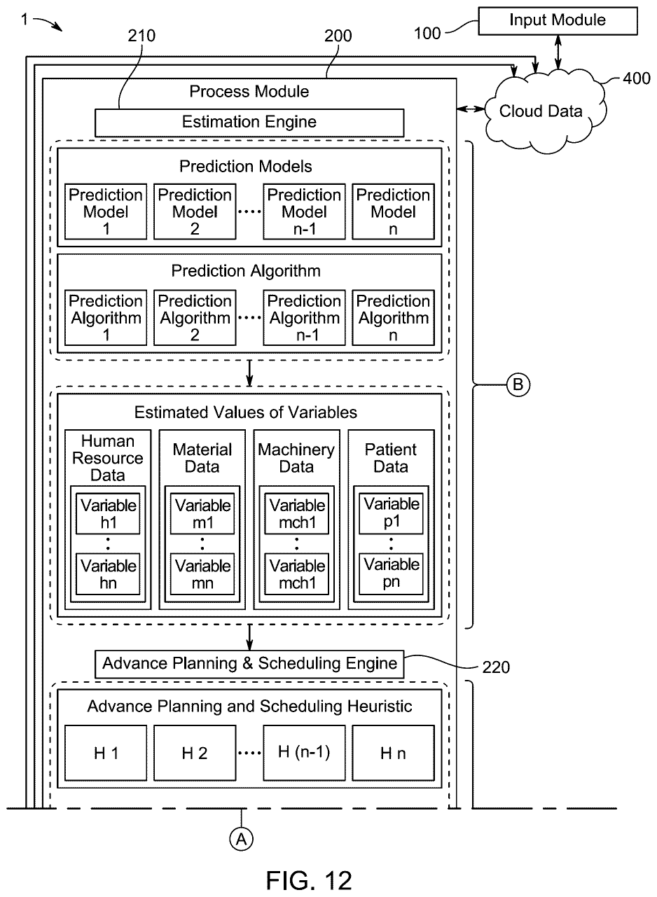

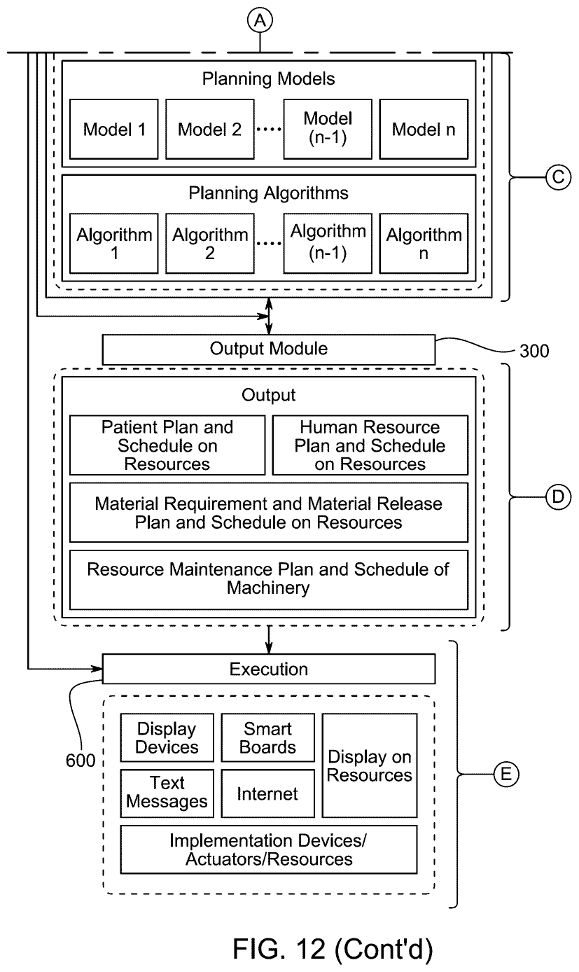

FIG. 12 illustrates schematic diagram of integration of input module 100, process module 200, execution mechanism 600 and cloud-based data storage 400 in the disclosed method and system 1. Input module 100 shares input data 100A with cloud-based data storage 400 using information sharing mechanism 500 which is offloaded by process module 200 utilizing information sharing mechanism 500. The process module 200 gets input data 100A of each variable which directly or indirectly influences the planning and scheduling of patients and resources in healthcare facilities.

The estimation engine 210 updates the prediction models and algorithms as per input data 100A of each variable using machine learning and other artificial intelligence techniques/methods. The estimation engine 210 runs the trained/updated prediction models and algorithms to predict and calculate the estimated values of variables of human resources, material, machinery and patients data and shares these estimated values of variables 219 with the advance planning and scheduling engine 220.

Advance planning and scheduling engine 220 runs advance planning and scheduling heuristics, models, and algorithms to generate patient plan and schedule on resources, human resource plan and schedule on resources, material requirement and material release plan and schedule and resource maintenance plan and schedule on resources in the healthcare facility. These plans are input to the output module 300 through cloud-based data storage 400 and communication mechanism 500.

In addition, process module 200 synchronizes all plans with the patient plan and schedule which is generated for various levels of planning hierarchy. The advance planning and scheduling engine 220 integrates all levels of planning hierarchy with feedback mechanism at all levels of the planning hierarchy and used push and pull rules for generation of feasible solutions for various iterations in the optimization algorithms. The process module 200 shares the processed information with the cloud data utilizing any kind of data sharing medium and also sends the processed data with the output module of the disclosure.

The output module 300 communicates and displays Patient Plan and Schedule on Resources with the execution, Human Resource Plan and Schedule on Resources with the execution, Material Requirement and Material Release Plan and Schedule on Resources with the execution, Resource Maintenance Plan and Schedule on Resources with the execution.

The execution mechanism 600 executes and implements these plans with all resources in the healthcare facility in real time utilizing actuators including but not limited to display devices, smart boards, display on resources, shared through internet, mobile phone messages. The execution mechanism 600 generates a work plan for various resources and displays and runs the work plan on computer systems in real time with all resources in the healthcare facility.

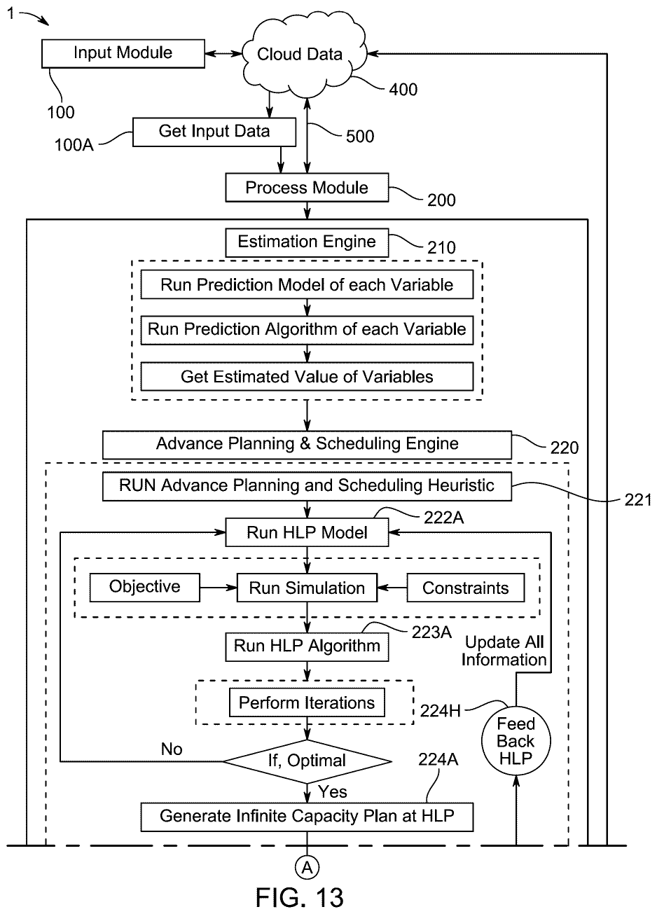

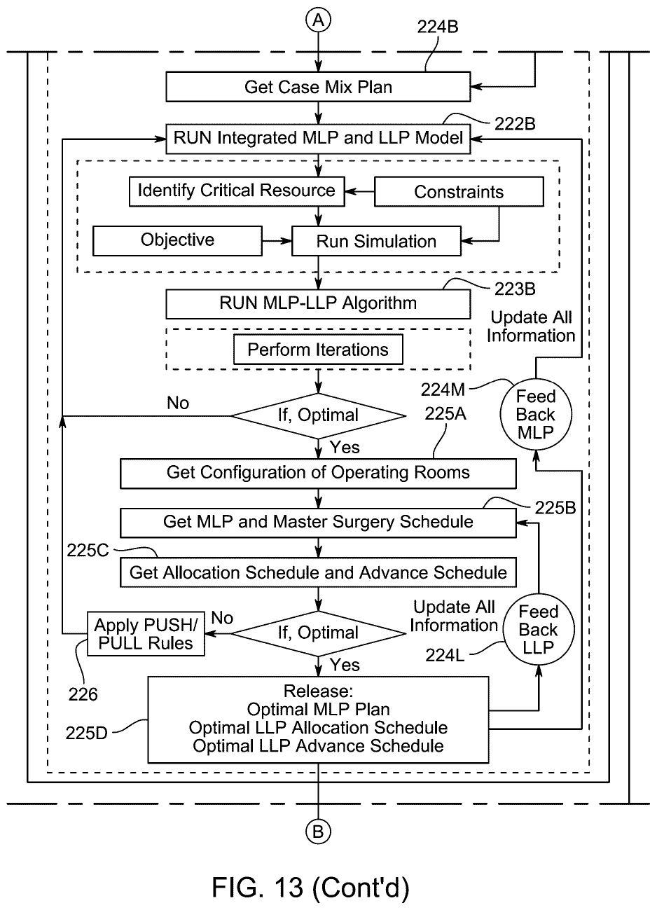

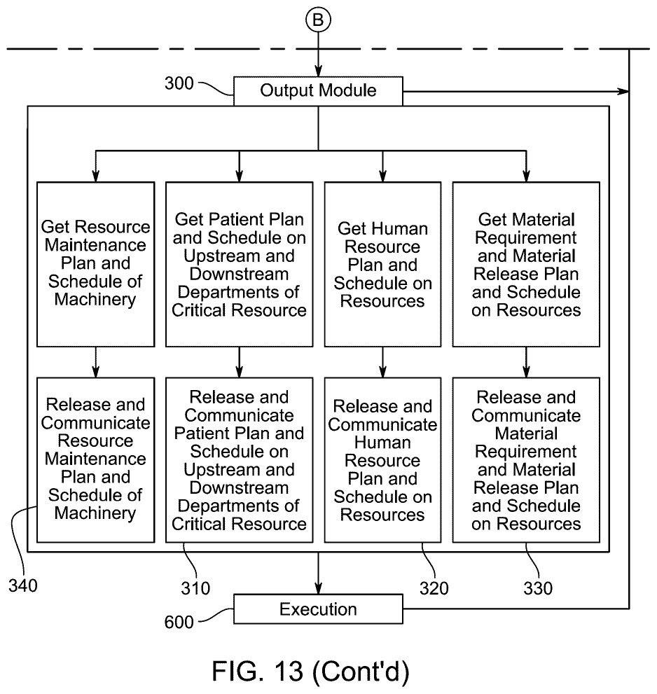

FIG. 13 illustrates systematic view of the system comprising of modules and the working of each module of system 1. The input module 100 shares input data 100A with the cloud-based data storage 400 through information sharing mechanism 500 which is offloaded in process module 200 using information sharing mechanism 500.

Estimation engine 210 runs the prediction models 211, runs prediction algorithms 216 and gets estimated values of the variables 219 which is shared with the advance planning and scheduling engine 220 using information sharing mechanism 500.

Advance planning and scheduling engine 220 utilizes multi-level planning of patients on identified critical resource of the planning period. The higher-level planning involves the planning of patients on each critical resource of the healthcare facility. At a higher level, the patients are allocated to the planning horizons based on their arrival, considering the infinite capacity of all critical resources. At the medium level, the patients from higher levels are allocated to their required critical resources, including the operating room, ICU, and ward. At the lower level, the respective planning and scheduling model is performed. The plan is then released and is communicated to other upstream departments and resources.

Advance planning and scheduling module 220 runs advance planning and scheduling heuristic 221, runs Higher-Level Planning (HLP) model 222A which is aimed for higher level planning and scheduling using any kind of mathematical, numerical, simulation models where constraints and objectives are defined and modeled. HLP model 222A has all possible constraints of human resources, patients, material and machinery of the healthcare facility. HLP model is solved by running HLP algorithm 223A to perform iterations to determine optimal solution of HLP plan and schedule considering any kind of optimality condition, or objective function of HLP model 222A including but not limited to maximize utilization of the resources in HLP planning periods in advance in the near future in the near coming month, week, day or other planning period scale or unit used in the healthcare facility. The optimal plan and schedule obtained after satisfying optimality condition at HLP is used to generate infinite capacity plan at HLP 224A and case mix plan 224B. Infinite capacity plan 224A and case mix plan 224B are communicated to HLP model 222A for planning and scheduling in the next planning period through feedback HLP 224H which is aimed in the advance planning and scheduling heuristic 221 to identify any capacity available in the planning periods at any stage of operation and can be filled with any kind of emergency arrival of patients in the healthcare facility. Moreover, feedback HLP 224H is used to deliver updated information of plan and schedule to the HLP model 223A for allocation of patients to different planning periods in dynamic environment in the healthcare facility.

The infinite capacity plan at HLP 224A and case mix plan 224B are used as input to run integrated Medium-Level Planning (MLP) and Lower-Level Planning (LLP) model 222B where different models with any type and kind of optimization objectives and constraints are used to identify the critical resource of the near future in the near coming month, week, day or other planning period scale or unit used in the healthcare facility. The models used in integrated MLP and LLP model 222B can be any type of mathematical model, numerical model and or simulation model in which various constraints relating to MLP and LLP plans are considered including but not limited to estimated values of status and condition of resources, estimated values of status and condition of staff/workers performing tasks on the machinery and/or resources, estimated values of status and condition of patients, estimated values of status and condition of various type of material which is used in the MLP and LLP operations to calculate the estimated/forecasted or most expected critical resource in the near future planning period.

The integrated MLP and LLP model 222B of the identified critical resource is run and solved using run MLP-LLP algorithm 223B to check for optimality condition and/or optimization objective/objectives of MLP and LLP plans and schedules including but not limited to maximize utilization of identified critical resource and other resources which are interlinked with the identified critical resource in MLP and LLP planning periods. MLP-LLP algorithm 223B identifies optimal configuration of operating rooms 225A, optimal MLP and master surgery schedule 225B, and optimal allocation schedule and optimal advance schedule 225C at MLP and LLP planning periods using PUSH/PULL rules 226. PUSH/PULL rules 226 include constraints or any kind of scheduling rules, conditions, constraints related to expected values variables 219 of human resource, machinery, material, patient priority rules, or any kind of rules, if or else conditions which can give feasible plan and schedule and also includes the integrated constraints between human resources, material, machinery, resources, patients which can influence on the plan and schedule of patients on resources, maintenance plan and schedule of machinery, material requirement and material release plan and schedule and human resource plan and schedule on resources. Run MLP-LLP algorithm 223B determines an optimal MLP and LLP plan and schedule to release optimal MLP plan, optimal LLP allocation schedule and optimal LLP advance schedule by solving integrated MLP and LLP model. Moreover, Run MLP-LLP algorithm 223B determines an optimal MLP master surgery schedule 225B and releases optimal MLP plan, optimal LLP allocation schedule, optimal LLP advance schedule 225D. Feedback MLP 224M is used to deliver updated information of optimal MLP plan to integrated MLP and LLP model 222B. Moreover, feedback LLP 224L, is used to deliver updated information of optimal LLP plan to MLP and master surgery schedule 225B. Feedback MLP 224M and feedback LLP 224L are aimed in the advance planning and scheduling heuristic 221 to identify any capacity available in the medium level planning periods and lower level planning periods at any stage of operation and can be filled with PUSH/PULL rules 226 from or any kind of emergency arrival of patients in the healthcare facility. Moreover, feedback MLP 224M and feedback LLP 224L are used to deliver updated information of plan and schedule to the integrated MLP and LLP model 222B for allocation of patients to different medium level planning periods and lower-level planning periods in dynamic environment in the healthcare facility. The release MLP and LLP plan 225D is shared with the output module 300.

Output module 300 is utilized to get resource maintenance plan and schedule of machinery, and release and communicate resource maintenance plan and schedule of machinery 340; get patient plan and schedule on upstream and downstream departments of the identified critical resource of the planning period and release and communicate patient plan and schedule on upstream and downstream departments of the identified critical resource 310; get human resource plan and schedule on resources and release and communicated human resource plan and schedule on resources 320; get material requirement and material release plan and schedule on resources and release and communicate material requirement plan and material release plan and schedule on resources 330. The output module 300 shares the plan and schedule 310, 320, 330, 340 with the cloud-based data storage 400 and with the execution mechanism 600.

The execution mechanism 600 executes and implements patient plan and schedule on upstream and downstream departments of the identified critical resource 310, human resource plan and schedule on resources and release and communicate human resource plan and schedule on resources 320, material requirement and material release plan and schedule on resources and release and communicate material requirement plan and material release plan and schedule on resources 330, resource maintenance plan and schedule of machinery, and release and communicate resource maintenance plan and schedule of machinery 340, with all resources in the healthcare facility in real time in dynamic environment utilizing any kind and type of actuators including but not limited to display devices, smart boards, display on resources, text messages, internet, and other kinds of implementation devices/actuators/resources.

FIG. 13 further illustrates detailed view of the disclosed method and system 1. In FIG. 13, get input data 100A contains the data from human resource including but not limited to: age of doctor, nurses, staff, their expected availability time, specialty, fatigue, recovery, learning, forgetting behavior; material data including not limited to: usage rate, availability, lead time, reorder, replenishment cycle, remaining life; machinery data including but not limited to: machine health and life, expected type of failures, expected time to failure, expected time to repair, repairing time, supplier; patient data including but not limited to: expected arrival and type of patients, expected number of patients arrival for each type, expected time of discharge for admitted patients, expected criticality of patients.

The input data is processed in process module 200 to determine human resource data including but not limited to: expected performance, expected rest time and duration of rest, expected duration for work, expected duration to perform different tasks, expected fatigue and recovery cycle, expected learning and forgetting cycle for various tasks; material data including but not limited to: expected usage in the forthcoming planning period, expected shortage, expected expiry, expected arrival time; machinery data including but not limited to: probability of failure of each type of failure, expected duration of different type of failures, expected duration of repair time for each type of failure, expected availability of tools, expected lead time; patient data including but not limited to; expected arrival and type of patients, expected number of patients arrival for each type, expected time of discharge for admitted patients, expected criticality of patients, expected time of stay on different resources; using estimation engine 210. The estimated value of variable 219 are used in the HLP model 222A to get near coming or look ahead planning period; get duration of HLP planning periods; calculate PST of patients, optimally insert patients in HLP planning periods and look ahead periods based on their constraints, PST, expected duration of stay on various resources and objective, generate infinite capacity plan of HLP 224A planning periods; generate optimal case mix plan 224B considering look ahead HLP planning period.

An infinite capacity plan 224A and case mix plan 224B are utilized in an integrated MLP and LLP model 222B. The integrated MLP and LLP model gets the duration of MLP and LLP planning periods; calculates required capacity for each specialty group of patients; calculates criticality of resources in MLP and Look ahead planning periods using workload, efficiency of resources, availability of resource, work in process, health, fatigue and recovery, learning-forgetting behavior of human resources, and health life of machinery and material availability and any other factor; identify the critical resource in forthcoming MLP and LLP planning periods; generate set of feasible MLP plans to distribute capacity of identified critical resource among specialty groups; generate set of feasible LLP schedules for each feasible MLP plan; run simulation model and optimization algorithm of identified critical resource to calculate objective of MLP and LLP; generate optimal operating room configurations 225A; generate optimal MLP and master surgery schedule 225B at MLP and its corresponding allocation schedule and advance schedule of patients 225C in each LLP of the considered MLP, send feedback 224M to MLP to update MLP plan and send feedback 224L to MLP master surgery schedule 225B. If capacity of resources is not fully utilized, apply PUSH and PULL Rules 226 and RUN integrated MLP and LLP model 222B. Otherwise, identify optimal MLP plan of MLP and look ahead MLP planning period/next planning periods and send feedback to HLP to update HLP plan 224M. Optimal MLP plan, optimal LLP allocation schedule and optimal LLP advance schedule 225D is shared with the output module 300.

Output module 300 gets and releases and communicates resource maintenance plan and schedule of machinery 340; gets and releases and communicates patient plan and schedule on upstream and downstream departments of critical resource 310; gets and releases and communicates human resource plan and schedule on resources 320; gets and releases material requirement and material release plan and schedule on resources 330; and shares with execution mechanism 600.

The execution mechanism 600 executes and implements patient plan and schedule on upstream and downstream departments of the identified critical resource 310 of the planning period, human resource plan and schedule on resources and release and communicated human resource plan and schedule on resources 320, material requirement and material release plan and schedule on resources and release and communicate material requirement plan and material release plan and schedule on resources 330, resource maintenance plan and schedule of machinery, and release and communicate resource maintenance plan and schedule of machinery 340, with all resources in the healthcare facility in real time in dynamic environment utilizing actuators including but not limited to display devices, smart boards, display on resources, text messages, internet, and other kind of implementation devices/actuators/resources.

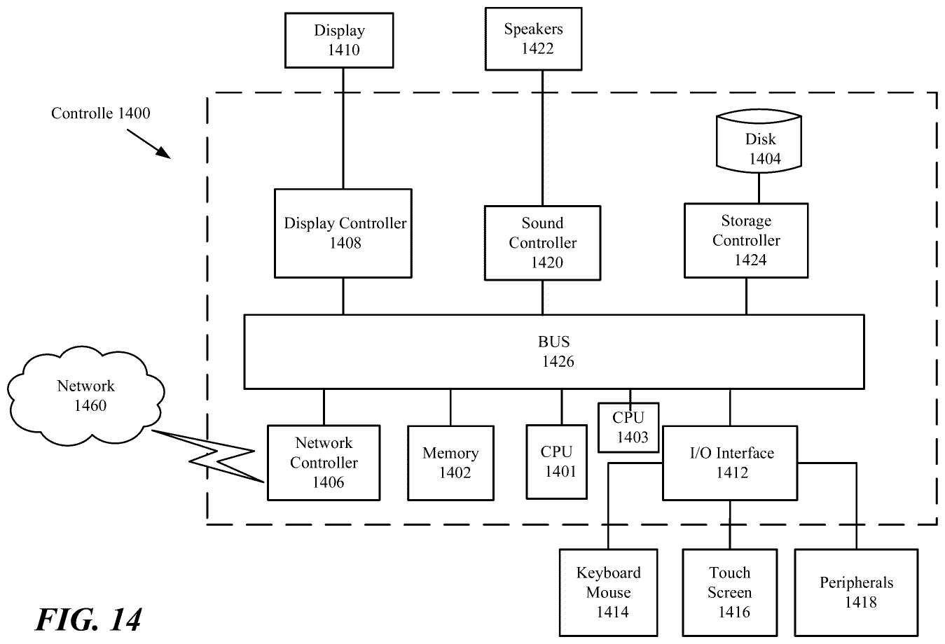

Next, further details of the hardware description of the computing environment according to exemplary embodiments is described with reference to FIG. 14. In FIG. 14, a controller 1400 is described is representative of the system in which the controller is a computing device which includes a CPU 1401 which performs the processes described above/below. The process data and instructions may be stored in memory 1402. These processes and instructions may also be stored on a storage medium disk 1404 such as a hard drive (HDD) or portable storage medium or may be stored remotely.

Further, the present disclosure is not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the present disclosure may be provided as a utility application, background daemon, or component of an operating system, or combination thereof, executing in conjunction with CPU 1401, 1403 and an operating system such as Microsoft Windows 7, Microsoft Windows 10, UNIX, LINUX, Apple MAC-OS and other systems known to those skilled in the art.

The hardware elements in order to achieve the computing device may be realized by various circuitry elements, known to those skilled in the art. For example, CPU 1401 or CPU 1403 may be a Xenon or Core processor from Intel of America or an Opteron processor from AMD of America, or may be other processor types that would be recognized by one of ordinary skill in the art. Alternatively, the CPU 1401, 1403 may be implemented on an FPGA, ASIC, PLD or using discrete logic circuits, as one of ordinary skill in the art would recognize. Further, CPU 1401, 1403 may be implemented as multiple processors cooperatively working in parallel to perform the instructions of the inventive processes described above.

The computing device in FIG. 14 also includes a network controller 1406, such as an Intel Ethernet PRO network interface card from Intel Corporation of America, for interfacing with network 1460. As can be appreciated, the network 1460 can be a public network, such as the Internet, or a private network such as an LAN or WAN network, or any combination thereof and can also include PSTN or ISDN sub-networks. The network 1460 can also be wired, such as an Ethernet network, or can be wireless such as a cellular network including EDGE, 3G, 4G, and 5G wireless cellular systems. The wireless network can also be WiFi, Bluetooth, or any other wireless form of communication that is known.

The computing device further includes a display controller 1408, such as a NVIDIA Geforce GTX or Quadro graphics adaptor from NVIDIA Corporation of America for interfacing with display 1410, such as a Hewlett Packard HPL2445w LCD monitor. A general purpose I/O interface 1412 interfaces with a keyboard and/or mouse 1414 as well as a touch screen panel 1416 on or separate from display 1410. General purpose I/O interface also connects to a variety of peripherals 1418 including printers and scanners, such as an OfficeJet or DeskJet from Hewlett Packard.

A sound controller 1420 is also provided in the computing device such as Sound Blaster X-Fi Titanium from Creative, to interface with speakers/microphone 1422 thereby providing sounds and/or music.

The general purpose storage controller 1424 connects the storage medium disk 1404 with communication bus 1426, which may be an ISA, EISA, VESA, PCI, or similar, for interconnecting all of the components of the computing device. A description of the general features and functionality of the display 1410, keyboard and/or mouse 1414, as well as the display controller 1408, storage controller 1424, network controller 1406, sound controller 1420, and general purpose I/O interface 1412 is omitted herein for brevity as these features are known.

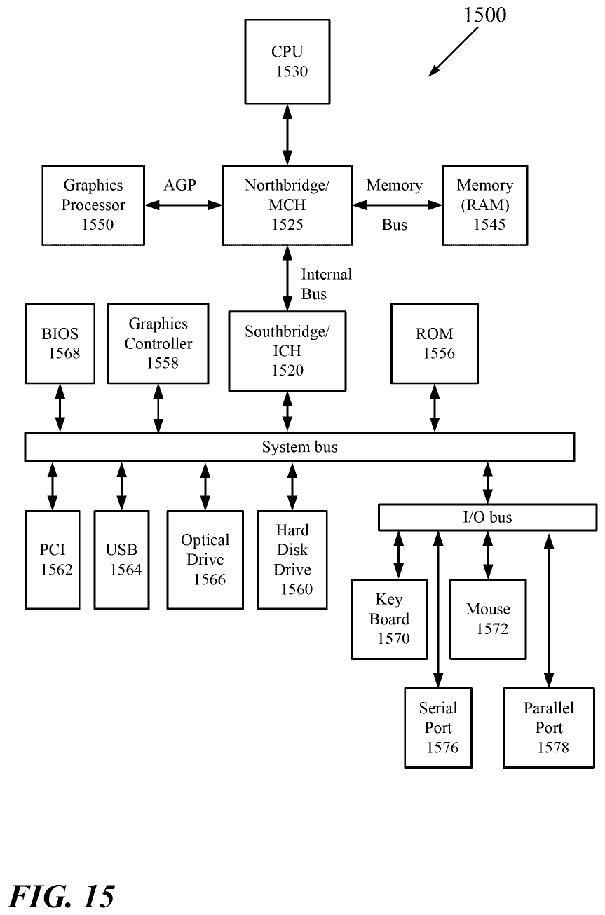

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 15.

FIG. 15 shows a schematic diagram of a data processing system, according to certain embodiments, for performing the functions of the exemplary embodiments. The data processing system is an example of a computer in which code or instructions implementing the processes of the illustrative embodiments may be located.

In FIG. 15, data processing system 1500 employs a hub architecture including a north bridge and memory controller hub (NB/MCH) 1525 and a south bridge and input/output (I/O) controller hub (SB/ICH) 1520. The central processing unit (CPU) 1530 is connected to NB/MCH 1525. The NB/MCH 1525 also connects to the memory 1545 via a memory bus, and connects to the graphics processor 1550 via an accelerated graphics port (AGP). The NB/MCH 1525 also connects to the SB/ICH 1520 via an internal bus (e.g., a unified media interface or a direct media interface). The CPU Processing unit 1530 may contain one or more processors and even may be implemented using one or more heterogeneous processor systems.

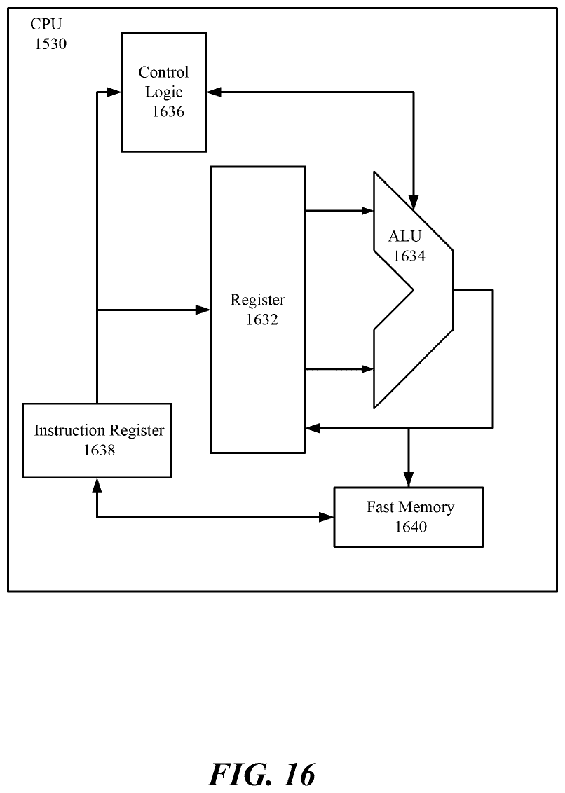

For example, FIG. 16 shows one implementation of CPU 1530. In one implementation, the instruction register 1638 retrieves instructions from the fast memory 1640. At least part of these instructions are fetched from the instruction register 1638 by the control logic 1636 and interpreted according to the instruction set architecture of the CPU 1530. Part of the instructions can also be directed to the register 1632. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 1634 that loads values from the register 1632 and performs logical and mathematical operations on the loaded values according to the instructions. The results from these operations can be feedback into the register and/or stored in the fast memory 1640. According to certain implementations, the instruction set architecture of the CPU 1530 can use a reduced instruction set architecture, a complex instruction set architecture, a vector processor architecture, a very large instruction word architecture. Furthermore, the CPU 1530 can be based on the Von Neuman model or the Harvard model. The CPU 1530 can be a digital signal processor, an FPGA, an ASIC, a PLA, a PLD, or a CPLD. Further, the CPU 1530 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 15, the data processing system 1500 can include that the SB/ICH 1520 is coupled through a system bus to an I/O Bus, a read only memory (ROM) 1556, universal serial bus (USB) port 1564, a flash binary input/output system (BIOS) 1568, and a graphics controller 1558. PCI/PCIe devices can also be coupled to SB/ICH 1588 through a PCI bus 1562.

The PCI devices may include, for example, Ethernet adapters, add-in cards, and PC cards for notebook computers. The Hard disk drive 1560 and CD-ROM 1566 can use, for example, an integrated drive electronics (IDE) or serial advanced technology attachment (SATA) interface. In one implementation the I/O bus can include a super I/O (SIO) device.

Further, the hard disk drive (HDD) 1560 and optical drive 1566 can also be coupled to the SB/ICH 1520 through a system bus. In one implementation, a keyboard 1570, a mouse 1572, a parallel port 1578, and a serial port 1576 can be connected to the system bus through the I/O bus. Other peripherals and devices that can be connected to the SB/ICH 1520 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry, or based on the requirements of the intended back-up load to be powered.

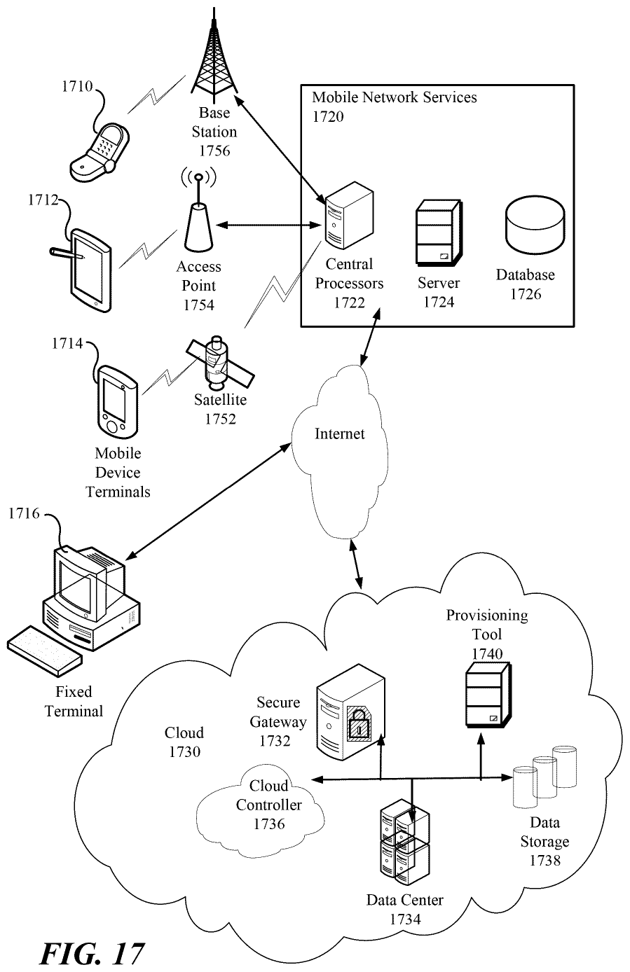

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 17, in addition to various human interface and communication devices (e.g., display monitors, smart phones, tablets, personal digital assistants (PDAs)). More specifically, FIG. 17 illustrates client devices including a smart phone 1711, a tablet 1712, a mobile device terminal 1714 and fixed terminals 1716. These client devices may be commutatively coupled with a mobile network service 1720 via a base station 1756, an access point 1754, a satellite 1752 or via an internet connection. The mobile network service 1720 may comprise central processors 1722, a server 1724 and a database 1726. The fixed terminals 1716 and the mobile network service 1720 may be commutatively coupled via an internet connection to functions in cloud 1730 that may comprise a security gateway 1732, a data center 1734, a cloud controller 1736, a data storage 1738 and a provisioning tool 1740. The network may be a private network, such as the LAN or the WAN, or may be the public network, such as the Internet. Input to the system may be received via direct user input and received remotely either in real-time or as a batch process. Additionally, some implementations may be performed on modules or hardware not identical to those described. Accordingly, other implementations are within the scope that may be disclosed.

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

1. An intelligent system for creating and releasing to a network of computers an integrated plan and schedule in a dynamic healthcare environment of a healthcare facility, comprising:
a healthcare facility computing network interconnecting the network of computers;
a healthcare facility data base management system maintaining historical data related to status and condition of healthcare provider resources, material, machinery and patients; and
a processor implemented as multiple processors cooperatively working in parallel to execute instructions configured to
retrieve the historical data from the healthcare facility data base management system, and data in real time, corresponding to a plurality of variables related to the status and condition of healthcare provider resources, material, machinery and patients,
train in parallel, by executing the instructions in each of the multiple processors, a respective machine learning model for each of healthcare provider availability prediction, material resource prediction, machinery availability probability prediction, and patient health data prediction, using machine learning based on the retrieved historical data, to predict values of each of the plurality of variables for a future time,
predict in parallel, by executing the instructions in each of the multiple processors the respective machine learning model for each of healthcare provider availability prediction, material resource prediction, machinery availability probability prediction, and patient health data prediction, using real time and updated historical data, the values of each of the plurality of variables for a future time, and
input the predicted values of the plurality of variables to an advance planning and scheduling engine,
wherein the advanced planning and scheduling engine is configured, with different simulation models that are subject to constraints and optimization objectives, to determine a critical resource among all resources of the healthcare facility in a planning period and determine in parallel, by executing the instructions in each of the multiple processors using the different simulation models, various feasible solutions of a planning and scheduling algorithm at each of multiple levels of a planning hierarchy based on a maximum utilization of the identified critical resource in the planning period, wherein the multiple levels of the planning hierarchy include higher level planning for the healthcare facility over the planning period, medium level planning for scheduling of the resources, and lower level planning for scheduling within each resource;
wherein the multiple processors work in parallel to execute the instructions further configured to
generate a material requirement plan and schedule, material release plan and schedule, machinery maintenance plan and schedule, and healthcare provider resource plan and schedule in advance,
synchronize the plans with a patient plan and schedule which is generated for the multiple levels of the planning hierarchy,
use push and pull rules to move patients between planning periods of the higher level planning for generation of feasible plans,
perform iterations of an optimization algorithm to search in the medium and the lower level planning an optimal patient plan, integrated with human resource plan, material plan, resources plan and resources maintenance plan,
generate the optimal patient plan and schedule of patients on resources integrated with maintenance planning and scheduling of machinery, material requirement planning, material release scheduling and human resource planning and scheduling in the dynamic environment of the healthcare facility to create an integrated plan and schedule,
release and execute the integrated plan and schedule in the interconnected computers in the computing network, and
update in parallel, by executing the instructions in each of the multiple processors, the respective machine learning model for each of healthcare provider availability prediction, material resource prediction, machinery availability probability prediction, and patient health data prediction, using machine learning.

2. The system of claim 1, further comprising a cloud-based data storage, wherein the processor stores solution-related data in the cloud-based data storage.

3. The system of claim 1, further comprising an output device,
wherein the processor is configured to send solution-related data to the output device.

4. The system of claim 1, further comprising
a plurality of display devices of each of the computers in the computing network configured to display a patient plan and schedule, a healthcare provider plan and schedule, a material resource plan and schedule, and a machinery maintenance plan and schedule.

5. The system of claim 1, wherein the multiple processors working cooperatively in parallel to execute the instructions further comprising:
performing the optimization algorithm configured to use the planning and scheduling models to determine an optimal patient plan and schedule on the critical resources and determine if capacity of each of the critical resources is available or it is fully utilized,
wherein for each critical resource the push and pull rules include,
if there is available capacity on the critical resource in a coming planning period, available capacity information is shared with the higher level planning using a feedback mechanism and the patients in a future planning period are pulled on the planning period after identifying feasibility based on constraints related to patients, healthcare provider resources, material, machines,
otherwise, if the capacity of the critical resources is not available, an excess number of patients are moved to the next planning period or pushed, and this movement information is shared with the higher-level planning using the feedback mechanism keeping in view the constraints related to patients, healthcare provider resources, material and machines in the healthcare facility of the medium and the low level planning.

6. The system of claim 3, wherein the output device is further configured to create a material requirement plan, a material release plan and schedule, and a healthcare provider resource plan and maintenance and schedule plan based on a critical resource in advance of time of planning period in the dynamic environment of the healthcare facility.

7. The system of claim 1, wherein the multiple processors working cooperatively in parallel to execute the instructions further comprising
an execution mechanism configured to obtain the optimal patient plan, the material requirement plan, the material release plan, the healthcare provider resource plan, the schedule and maintenance plan and a schedule of machinery in real time and execute these plans using a combination of smart displays and smart boards, and transmit the plans through mobile phone messages.

8. The system of claim 1, wherein the multiple processors working cooperatively in parallel to execute the instructions for the optimization algorithm further configured to identify critical resources and use the planning and scheduling models to further calculate
an optimal configuration of operating rooms, and
an optimal master surgery schedule.

9. The system of claim 1, wherein the multiple processors working cooperatively in parallel to execute the instructions is further configured to:
obtain real time condition data of human resources, including health status, experience, learning, forgetting, fatigue, tasks performance status, and tasks performance attitude, of doctors, nurses, and other human resource involved directly or indirectly in operations of the healthcare facility, and
predict an expected future availability or unavailability of the human resources for specific tasks based on the real time condition data of the human resources.

10. The system of claim 1, wherein the multiple processors working cooperatively in parallel to execute the instructions is further configured to:
predict health life and expected failure of the machinery at the healthcare facility, and
determine an expected failure type and expected time of failure which is used in preparing the machine maintenance plan and schedule.

11. A computer-implemented method for creating and releasing to a network of computers an integrated plan and schedule in a dynamic healthcare environment of a healthcare facility having a healthcare computing network interconnecting the network of computers, comprising:
obtaining in real time and retrieving historical data, from a healthcare facility data base management system, corresponding to a plurality of variables related to status and condition of healthcare provider resources, material, machinery and patients;
training in parallel, by executing instructions in each of multiple processors, a respective machine learning model for each of healthcare provider availability prediction, material resource prediction, machinery availability probability prediction, and patient health data prediction, using machine learning based on the retrieved historical data, to predict values of each of the plurality of variables for a future time;
predicting in parallel, by executing instructions in each of the multiple processors the respective machine learning model for each of healthcare provider availability prediction, material resource prediction, machinery availability probability prediction, and patient health data prediction, using real time and updated historical data, the values of each of the plurality of variables for a future time;
inputting the predicted values of the plurality of variables to an advance planning and scheduling engine, wherein the advanced planning and scheduling engine is configured, with different simulation models that are subject to constraints and optimization objectives;
determining in parallel, by the multiple processors, various feasible solutions of the simulation models of the planning and scheduling algorithm at various levels of a planning hierarchy;
generating a material requirement plan and schedule, material release plan and schedule, machinery maintenance plan and schedule, and healthcare provider resource plan and schedule in advance;
synchronizing the plans with a patient plan and schedule which is generated for each of multiple levels of the planning hierarchy, wherein the multiple levels of the planning hierarchy include higher level planning for the healthcare facility over the planning period, medium level planning for scheduling of the resources, and lower level planning for scheduling within each resource;
using push and pull rules to move patients between planning periods of the higher level planning for generation of feasible plans;
performing iterations of an optimization algorithm to search in the medium and the lower level planning an optimal patient plan, integrated with a human resource plan, a material plan, a resources plan and a resources maintenance plan;
generating the optimal patient plan and schedule of patients on resources integrated with maintenance planning and scheduling of machinery, material requirement planning, material release scheduling and human resource planning and scheduling in the dynamic environment of healthcare facility to create an integrated plan and schedule;
releasing and executing the integrated plan and schedule in the interconnected computers in the computing network; and
updating in parallel, by executing the instructions in each of the multiple processors, the respective machine learning model for each of healthcare provider availability prediction, material resource prediction, machinery availability probability prediction, and patient health data prediction, using machine learning.

12. The method of claim 11, further comprising storing solution-related data in a cloud-based data storage.

13. The method of claim 11, further comprising sending solution-related data to an output device.

14. The method of claim 11, further comprising displaying, by a plurality of display devices of each of the computers in the computing network, a patient plan and schedule, a healthcare provider plan and schedule, a material resource plan and schedule, and a machinery maintenance plan and schedule.

15. The method of claim 11, further comprising:
using the planning and scheduling dedicated model and the critical resources to determine an optimal patient plan; and
determining if capacity of each of the critical resources is available or it is fully utilized,
wherein for each critical resource the push and pull rules include,
if there is available capacity on the critical resource in a coming planning period, available capacity information is shared with the higher planning using a feedback mechanism and the patients in a future planning period are pulled on the planning period after identifying feasibility based on constraints related to patients, healthcare provider resources, material, machines,
otherwise, if the capacity of the critical resources is not available, an excess number of patients are moved to the next planning period or pushed, and this movement information is shared with the higher-level planning using the feedback mechanism keeping in view the constraints related to patients, healthcare provider resources, material and machines in the healthcare facility of the medium and the low level planning.

16. The method of claim 13, wherein
creating, by the output device, a material requirement plan, a material release plan and schedule, and a healthcare provider resource plan and maintenance and schedule plan based on a critical resource in advance of time of planning period in the dynamic environment of the healthcare facility.

17. The method of claim 11, further comprising
obtaining, by an execution mechanism, the optimal patient plan, the material requirement plan, the material release plan, the healthcare provider resource plan, the schedule and maintenance plan and a schedule of machinery in real time; and
executing the plans using a combination of smart displays and smart boards, and transmitting the plans through mobile phone messages.

18. The method of claim 11, further comprising:
identifying, by the optimization algorithm, critical resources; and
calculating, using the planning and scheduling models,
an optimal configuration of operating rooms, and
an optimal master surgery schedule.

19. The method of claim 11, further comprising:
obtaining real time and historical data, from a healthcare facility data base, including
real time condition data of human resources, historic data of condition of human resources, including health status, experience, learning, forgetting, fatigue, tasks performance status, and tasks performance attitude, of doctors, nurses, and other human resource involved directly or indirectly in operations of the healthcare facility; and
predicting an expected future availability or unavailability of the human resources for specific tasks based on the real time condition data of the human resources.

20. The method of claim 11, further comprising:
predicting a failure of the machinery at the healthcare facility; and
determining an expected failure type and expected time of failure which is used in preparing the machine maintenance plan and schedule.
